(19) United States
(12) Patent Application Publication
(10) Pub. No.: US 2022/0054582 A1
(43) Pub. Date: Feb. 24, 2022

(54) METHOD FOR MANUFACTURING A SOLID FORMULATION FOR ORAL ADMINISTRATION, ASSOCIATED FACILITY AND SOLID FORUMULATION

(71) Applicant: FERRING B.V., Hoofddorp (NL)

(72) Inventors: Robert RÖNNBACK, Morges (CH); Ludovic LECLERC, Romanel-sur-Lausanne (CH)

(73) Assignee: FERRING B.V., Hoofddorp (NL)

(21) Appl. No.: 17/283,178

(22) PCT Filed: Oct. 11, 2019
(86) PCT No.: PCT/EP2019/077663

§ 371 Date: Oct. 25, 2021

(30) Foreign Application Priority Data
Oct. 11, 2018 (FR) 1859441

(51) Int. Cl. A61K 38/095; A61K 9/00; A61K 47/38; B41F 17/00; B65B 9/02; B65B 35/44; B65B 57/14; B65B 57/18; B65B 61/06

(52) U.S. Cl. CPC A61K 38/095; A61K 9/0056; A61K 47/38; B41F 17/00; B65B 61/06; B65B 35/44; B65B 57/145; B65B 57/18; B65B 9/026

(57) ABSTRACT

Disclosed are methods and apparatuses for manufacturing a solid formulation for oral administration, comprising an edible substrate portion on which a composition comprising a first pharmaceutical active ingredient is deposited, and solid formulations made thereby. The methods comprise providing an edible substrate in the form of a film comprising a plurality of portions, spraying a liquid formulation comprising the active ingredient and a solvent onto an edible substrate portion and evaporating the solvent to form a solid composition deposited on the edible substrate portion; measuring the quantity of active ingredient supported by the edible substrate portion and comparing to a predetermined value, and dividing the edible substrate into the plurality of portions.

The present invention relates to a method for manufacturing a solid formulation for oral administration, said solid formulation comprising an edible substrate portion on which a first composition is deposited comprising a first pharmaceutical active ingredient, said method comprising the following steps: providing the edible substrate in film form; providing a first liquid formulation comprising the first pharmaceutical active ingredient and a solvent; then spraying the first liquid formulation on the film and evaporating the solvent, so as to form the first solid composition deposited on the substrate.

The invention particularly applies to solid formulations comprising an orodispersible film.

Oral pharmaceutical forms of orodispersible films are in particular known from documents EP 3,281,625 and US2017216220. Such pharmaceutical forms are in particular made by spreading, on a support, a solution comprising a pharmaceutical active ingredient and polymers, and drying the solution to obtain a film.

Such embodiments are suitable for producing large quantities of medicinal drugs, with uniform dosing. However, it would be interesting to produce this type of medicinal drug in a customized manner, for example by adapting the dosage to each patient.

To that end, experiments in producing oral pharmaceutical forms have been done, by depositing the active ingredient by inkjet-type printing on the substrate. Such experiments are in particular described in the following documents: Sandler et al., Inkjet Printing of Drug Substances and Use of Porous Substrates-Towards Individualized Dosing Journal of Pharmaceutical Sciences 2011, Vol. 100, 3396-3395; Genina et. al., Behavior of printable formulations of loperamide and caffeine on different substrates—effective print density in inkjet printing, Int. J. Pharm. 2013,453(2), 488-97; Alomari et. al., Personalized dosing: Printing a dose of one's own medicine, International Journal of Pharmaceutics 2015, 494, 568-577.

One technical problem related to the industrial production of oral pharmaceutical forms by printing is in particular controlling the dose of active ingredient in each produced pharmaceutical form.

Document EP3241539 A1 illustrates an earlier art of the above type. This document does not disclose a non-destructive measurement of a quantity of active ingredient, let alone a comparison of that quantity (which is not measured) to a reference value.

Document EP3403643 A1 relates in particular to the design of compositions containing an active ingredient. It discloses measurements made on prototypes of such compositions in order to validate their contents. However, it does not disclose a measurement that would be performed on an active ingredient contained in the result of a spraying of such a composition.

The present invention aims to allow the customized industrial production of medicinal drugs for oral administration. Tests that were carried out show that the medicines thus obtained have a stability (and therefore a shelf life) of at least six months.

The present invention involves spraying liquids onto surfaces in order to obtain the desired compositions by drying. It involves controlling the quantities sprayed. For convenience, the expressions “predetermined quantity” and “reference quantity” are used interchangeably to designate a target quantity (the quantity sought to be obtained), and such a quantity is also referred to as a “value” (“predetermined value” and “reference value” being synonymous with “predetermined quantity” and “reference quantity”).

The invention relates to a manufacturing method of the aforementioned type, and more particularly a method for the manufacture of a solid formulation for oral administration, said solid formulation comprising an edible substrate portion on which a first composition comprising a first pharmaceutical active ingredient is deposited, said method comprising the following steps:
providing the edible substrate in the form of a film;
providing a first liquid formulation comprising the first pharmaceutical active ingredient and a solvent; then
spraying the first liquid formulation onto the film and evaporating the solvent so as to form the first solid composition deposited on the substrate.

Said manufacturing method further comprises the following steps:
dividing (e.g. mechanically cutting, laser cutting or otherwise cutting) the film into a plurality of portions, such that each portion supports a quantity (which may be noted Q for a given portion) of first active ingredient corresponding to a predetermined value (which may be noted Q_{ref}); i.e. the spraying according to the method is adjusted so that the resulting quantity Q will be as close as possible to a predetermined desired value Q_{ref} (i.e. it should “correspond” to this predetermined value Q_{ref}); and
after the spraying step (and, according to an advantageous embodiment, after drying of the sprayed formulation), measuring the quantity of first active ingredient supported by each portion and comparing (this measured quantity) with the predetermined value; said measurement not destroying the solid formulation (those skilled in the art can choose any conventional non-destructive measurement method from among those available, including those mentioned in the description that follows).

Said comparison allows triggering an action (or several actions). It enables corrective measures to be taken, such as the destruction of portions in which the quantity of active ingredient is too far from the predetermined value, or, if the quantity is insufficient, the spraying of an additional quantity on the same portion in order to avoid having to destroy it. Said comparison also makes it possible (both alternatively and cumulatively) to check the spraying tuning and, if necessary, either to signal a tuning problem (for example by triggering an alert via an audible or visual alarm, or by sending an e-mail alert), or to make an automatic adjustment of the tuning, or both, i.e. to both signal a tuning problem and to adjust it automatically. This comparison is performed by an electronic device, such as a processor, microcontroller, on-board computer, or dedicated electronics.

According to another advantageous aspect of the invention, the method includes one or more of the following features, considered alone or according to any technically possible combinations:
a step of reporting a problem with the spraying tuning when the comparison of the measured quantity with the reference value reveals an abnormal deviation (this reporting is done by an electronic device, such as a processor, a microcontroller, an on-board computer, or dedicated electronics);
a step of adjusting the spraying tuning according to said comparison (this adjustment being carried out by an electronic device, such as a processor, a microcontroller, an on-board computer, or dedicated electronics);
the step of comparing with the predetermined value leads to classifying the film portions into two groups, the method next comprising a sorting step in which the two groups are separated from one another;
the first liquid formulation has a viscosity between 2 mPa·s and 20 mPa·s; and/or a surface tension between 20 mN/m and 50 mN/m, or, according to a specific embodiment, even between 25 mN/m and 50 mN/m;
the first active ingredient of the first liquid formulation is chosen from among analgesics, antihistamines, anti-inflammatories, antiepileptics and natural, synthetic or biotechnologically produced hormones;
according to a possible embodiment, the first liquid formulation further includes a dye;
the edible substrate comprises one or several hydrolysable polymers, preferably one or several cellulose derivatives,
a step of spraying the first liquid formulation over a first zone of each film portion, the method further comprising the following steps: providing a second liquid formulation comprising a second solvent; said second liquid formulation preferably comprising a second pharmaceutical active ingredient and/or a flavoring, then spraying the second liquid formulation on a second zone of the film and evaporating the second solvent, so as to form a second solid composition deposited on the substrate, the first and second zones of the film being separate from one another.

The invention further relates to a solid formulation for oral administration, derived from a method as described above, said formulation comprising an edible substrate portion on which at least one composition is deposited comprising a pharmaceutical active ingredient.

The invention further relates to an apparatus for the continuous manufacture of a solid formulation for oral administration, capable of implementing a method as described above, said apparatus comprising: a device for providing the edible substrate in the form of a strip of film, said strip being configured to be divided into several portions (whether by a complete or a discontinuous cutting of each portion, a discontinuous cutting, e.g. in the form of a dotted pre-cut, allowing such a portion to be easily separated at a later stage); a device for printing the first liquid formulation; an inspection device connected to the printing device, said inspection device being able to implement the printing of a predetermined quantity of first liquid formulation on each substrate portion; and a nondestructive measuring device for a quantity of first active ingredient supported by each portion; the inspection device being provided with means for comparing the measured quantity with a predetermined value (corresponding to the predetermined quantity, the mathematical “value” representing the physical “quantity” and designating the same reality) of first liquid formulation.

According to one advantageous aspect of the invention, the manufacturing apparatus further comprises an individual packaging device for each solid formulation.

The invention will be better understood upon reading the following description, provided solely as a non-limiting example and done in reference to the drawings, in which:

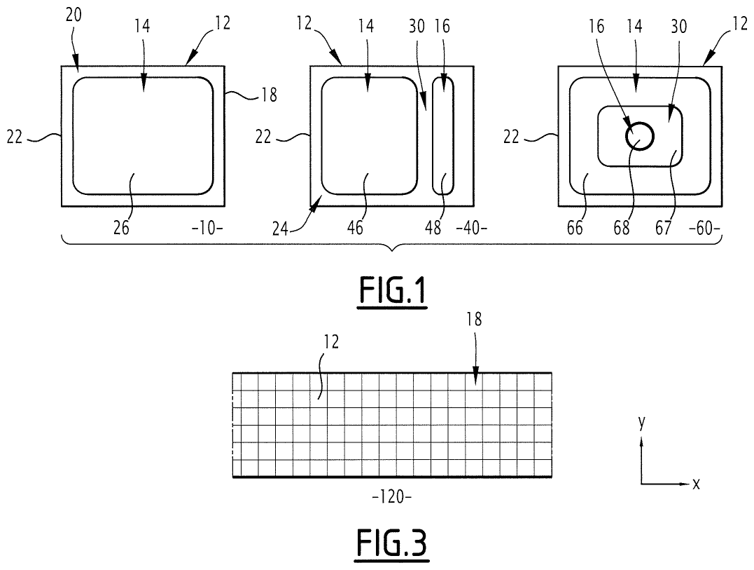

FIG. 1 is a schematic illustration of solid formulations according to a first, second and third embodiment of the invention;

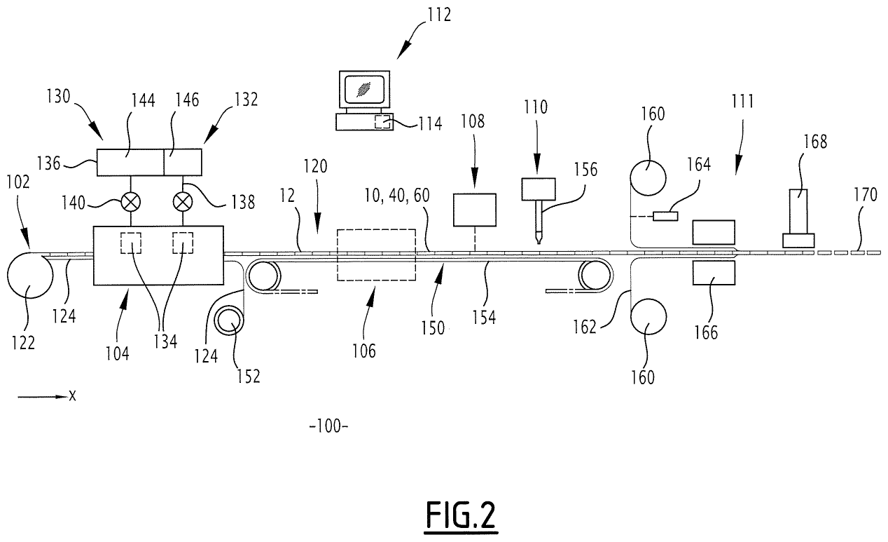

FIG. 2 is a schematic illustration of a manufacturing apparatus according to one embodiment of the invention, making it possible to produce the solid formulations of FIG. 1;

FIG. 3 is a detail view of an edible substrate that can be used in the apparatus of FIG. 2; and

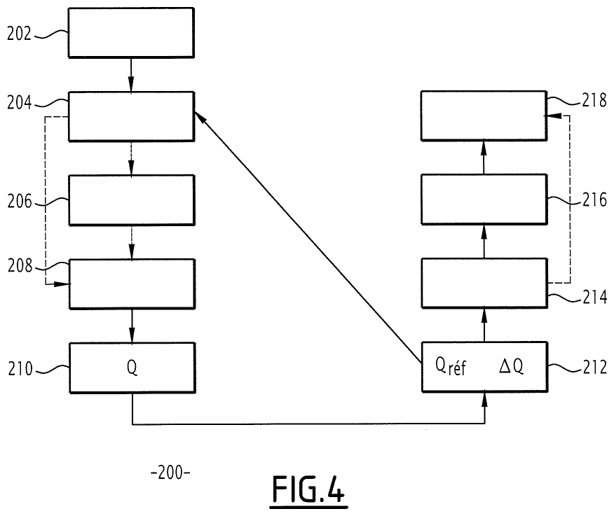

FIG. 4 is a logic diagram showing the steps of a manufacturing method, according to one embodiment of the invention, of the solid formulations of FIG. 1.

FIG. 1 shows a first 10, second 40 and third 60 solid formulation for oral administration, respectively according to a first, second and third embodiment of the invention.

The first 10, second 40 and third 60 solid formulations will be described simultaneously hereinafter, the common elements being designated by the same reference numbers.

These solid formulations are obtained by spraying. According to a possible embodiment, spraying is driven by a spraying control, which receives an input (or “tuning value” or “tuning quantity”).

According to a possible embodiment, the initial tuning value is set to a predetermined value. This means that this tuning value is, at the beginning of the method, equal to a predetermined quantity (value) that is to be sprayed.

According to an alternative and advantageous embodiment, the initial tuning value is only indexed on this predetermined value in order to be representative of it. Indeed, it is not important to use the same numerical value for the initial tuning and for the predetermined value, all that matters is to enable the interpretation of the numerical value used as tuning value in order to be able to determine that its initial value, whatever it may be, corresponds to the predetermined value and to interpret its subsequent value.

For example, when spraying is implemented using a printing technique, the sprayed quantity is, according to a possible embodiment, determined by a spraying tuning equal to the resolution (which may be noted RES) of the printing. For a given area to be printed, each resolution corresponds to a sprayed quantity and vice versa. The higher the resolution, the more dots are printed, and therefore the larger the quantity sprayed. The print resolution can be tuned in number of dots printed per distance unit (e.g. in dots per inch, DPI), whereby the same resolution can be used in both printing directions (in 2D printing). In this example, the spraying tuning is then a RES parameter (such as a resolution of 350 DPI), this parameter corresponding to a predetermined quantity to be sprayed.

In an improved version of this example, the tuning includes as an option (in addition to a RES resolution) a number NBP of passes. Thus, the process prints NBP-1 times a same area with maximum resolution and prints an NBPth time this same area with the RES resolution specified in the tuning, in order to increase the maximum sprayable quantity. In this case, the spraying tuning is a pair of parameters {RES, NPB} with NBP≥2 (the case NBP=1 being equivalent to the example of the previous paragraph, which does not define a number of passes and implements only a single pass), this pair corresponding to a predetermined quantity to be sprayed.

According to another example, the spraying tuning is a number N of bits (i.e. a number that can take 2^{N} values between 0 and 2^{N-1}, for example 1024 values between 0 and 1023 for N=10) initially representing the predetermined value. For example, if the predetermined value is likely to fluctuate in a range of values between VPₘᵢₙ (e.g. VPₘᵢₙ=10 mg) and VPₘₐₓ (e.g. VPₘₐₓ=2000 mg), the tuning for a predetermined value VP can be initially set to the integer closest to the value (2^{N-1})*(VP−VPₘᵢₙ)/(VPₘₐₓ−VPₘᵢₙ). For example, with the above illustrative numerical values, a predetermined value of 400 mg would result in an initial spraying tuning of:

1023*(400−10)/(2000−10)=200, i.e. 0xC8h in hexadecimal and 0x11001000b in binary. A value N greater than 10 allows the spraying tuning to be represented with a fairly fine accuracy. The lower the N value, the less accurate the tuning, the higher the N value, the more accurate the tuning.

The solid formulation 10, 40, 60 includes a substrate 12 and a first composition 14 deposited on said substrate. In the second and third embodiments, the solid formulation 40, 60 further includes a second composition 16 deposited on the substrate 12.

The substrate 12 is an edible substrate, assuming the form of a film 18. Preferably, the substrate 12 is an orodispersible substrate. Preferably, the substrate 12 does not contain any active ingredients. Preferably, the substrate 12 has a pH adapted to the active ingredient, so as to improve the conservation of the active ingredient over time. According to a possible embodiment, the substrate 12 is obtained from one or more pH-modifying excipient(s), such as citric acid or diluted phosphoric acid (as illustrated below in the formulation examples), to obtain an acidic pH. Other excipients may be used to obtain a basic pH.

More preferably, the substrate 12 comprises one or several water-soluble polymers. Still more preferably, said water-soluble polymers are chosen from among: cellulose derivatives, such as hydroxy propyl cellulose (HPC), hydroxypropyl methylcellulose (HPMC), carboxymethylcellulose sodium (CMC-Na) or hydroxyethyl cellulose (HEC); polyvinyl derivatives such as polyvinyl pyrrolidone (PVP), polyvinyl acetate (PVA) or PVP-PVA copolymers; natural gums such as xanthan gums or gums Arabic; alginates, in particular sodium alginates; carrageenan gums, such as iota or lambda carrageenans; starches, such as cornstarch, pea starch, or pregelatinized starches; pectins with high degrees of esterification; type B gelatins; dextrins; pullulans.

Still more preferably, the substrate 12 comprises one or several cellulose derivatives such as hydroxypropyl cellulose, hydroxypropyl methylcellulose, carboxymethylcellulose sodium or hydroxyethyl cellulose.

Optionally, the substrate 12 further comprises a flavoring and/or a sweetener, natural like fructose or synthetic like potassium acesulfame.

Optionally, the substrate 12 further comprises a saliva stimulant, such as citric or malic acid; or talcum (Mg₃Si₄O₁₀(OH)₂) and/or a dye such as titanium oxide (not preferred due to its possible carcinogenicity).

The film 18 forming the substrate 12 preferably has a thickness between 80 μm and 500 μm, and preferably between 80 μm and 120 μm, but, according to a possible embodiment, between 200 μm and 500 μm. According to a possible embodiment, the film 18 is designed to dissolve in a patient's mouth in less than three minutes, or, in an advantageous embodiment, in less than 30 seconds.

The substrate 12 includes a first 20 and second opposite face, delimited by a contour 22. Only the first face 20 is visible in FIG. 1.

The contour 22 defines a surface 24 of the substrate 12. Preferably, the surface 24 is chosen so as to allow the oral absorption in a single administration of the solid formulation 10, 40, 60. The surface 24 is for example between 0.5 cm² and 10 cm². In the embodiments shown in FIG. 1, the contour 22 has a square shape, the sides of which are about 2 cm long. Alternatively, the contour 22 may adopt other shapes, for example rectangular, polygonal, round or oval.

The surface 24 includes a first zone 26, 46, 66 on which the first composition 14 is deposited.

The first composition 14 in particular comprises a first pharmaceutical active ingredient. The invention primarily relates to active ingredients with relatively low concentrations, so as to be able to be achieved by spraying a small quantity of liquid composition on a substrate. Likewise, the invention primarily relates to compounds soluble in water or in relatively non-toxic solvents. According to a possible embodiment, the first composition is limited to the first active ingredient, excluding any other product (excipient, etc.).

The first active ingredient is for example an analgesic, an antihistamine, an anti-inflammatory, an antiepileptic or a natural, synthetic or biotechnologically-produced hormone.

The first active ingredient can be chosen from among a wide range of compounds including both small and large molecules. Preferably, the first active ingredient is a peptide such as desmopressin (1-desamino-8-d-arginine vasopressin).

Optionally, the first composition 14 also comprises a first dye, which makes it possible to see, with the naked eye, its deposition on the substrate 12 and which also allows, upstream, a measurement by colorimetry. The first dye is for example chosen from among food dyes, such as Erythrosine or Sunset Yellow.

The first zone 26, 46, 66 is arranged on the first face 20 of the substrate 12. In the first and second embodiments, the first zone 26, 46 has a square or rectangular shape. In the third embodiment, the first zone 66 has a substantially annular shape, delimiting a central space 67. Other shapes can be considered for the first zone, said shapes for example corresponding to a visual depiction or a decorative pattern.

Each of the second and third solid formulations 40, 60 further includes a second zone 48, 68 on which the second composition 16 is deposited.

The second composition 16 for example comprises a second pharmaceutical active ingredient and/or a flavoring, said flavoring for example being chosen from among the flavorings used in the food industry. Optionally, the second composition 16 also comprises a second dye, which makes it possible to view, with the naked eye, its deposition on the substrate 12.

The first 46, 66 and second 48, 68 zones are preferably separate from one another, i.e., not superimposed. More preferably, a space 30 is arranged between said first 46, 66 and second 48, 68 zones. This is advantageous, in that it avoids migration of products between different areas (their possible mixing could otherwise lead to a reduction of the shelf life).

According to one embodiment, like the second formulation 40, the first 46 and second 48 zones are arranged side by side.

According to another alternative embodiment, the first and second zones are arranged one around the other. In particular, in the third solid formulation 60, the second zone 68 is arranged in the central space 67 delimited by the first zone 66.

In the second and third solid formulations 40, 60 shown in FIG. 1, the second zone 48, 68 is located on the first face 20 of the substrate 12, as is the first zone 46, 66. In an alternative that is not shown, the first and second zones are arranged on two opposite faces of the substrate 12.

The deposition of the first 14 and second 16 compositions on two zones separated from one another makes it possible to eliminate the risk of chemical interaction between said compositions, which improves the stability of the solid formulation 40, 60. In an alternative that is not shown, the solid formulation includes at least three compositions, preferably deposited on three distinct zones of the substrate.

As will be described hereinafter, each of the first 14 and second 16 compositions is formed by spraying microdroplets of a liquid formulation comprising a solvent on the substrate 12, then by evaporation of said solvent. The first 14 and second 16 compositions therefore comprise all of the components of the corresponding liquid formulations, with the exception of the volatile components.

According to an advantageous embodiment, the spraying is carried out by a piezoelectric device. Alternatively, a thermal device can be used, for example. In the latter case, however, only active ingredients that cannot be degraded by the heating of the thermal device can be used, and the spraying is often less precise.

“Microdroplets” refer to drops with a size that may vary between about 10⁻¹² L and about 10⁻⁶ L.

FIG. 2 shows an apparatus 100 making it possible to manufacture a solid formulation 10, 40, 60 as described above.

The apparatus 100 comprises a set of juxtaposed devices, each of said devices allowing the implementation of a step of a method for manufacturing the solid formulation 10, 40, 60. Such a method will be described later.

The apparatus 10 in particular comprises: a device 102 for providing a film 18; a printing device 104; a drying device 106; an analysis device 108; a sorting device 110 and a packaging device 111. According to a possible embodiment, the film 18 is obtained by hot extrusion or 3D printing of a polymer resin. The apparatus 100 further comprises an electronic control unit 112, such as a computer, connected to said devices. The control unit 112 contains a program 114 making it possible to carry out the manufacturing method described later.

The provision device 102 includes a strip 120 of film 18, partially shown in FIG. 3. In an alternative embodiment, a device for manufacturing the film 18 is directly connected to the provision device 102. According to a possible implementation of this alternative embodiment, the manufacturing device implements the step of dividing the film 18 into a plurality of portions by manufacturing a divided film (e.g. a pre-cut film).

The film 18 is for example obtained by hot extrusion of a polymer resin, or by spreading such a resin on a support, followed by drying.

In the apparatus 100 of FIG. 2, the strip 120 is wound around itself around an axis in the form of a roller 122. Preferably, the roller 122 further includes a support tape 124 co-wound with the strip 120. The provision device 102 further includes means for emptying the roller 122.

According to an alternative that is not shown, the roller is replaced by a set of sheets distributed by a loader.

It will be considered that the unwound strip 120 extends primarily along a longitudinal direction X, as well as along a transverse direction Y. In the continuation of the description, the terms “inlet”, “outlet”, “upstream”, “downstream” relating to the apparatus 100 extend along the longitudinal direction X.

In the embodiment shown in FIGS. 2 and 3, the strip 120 is precut into portions 12, each portion being intended to form the substrate 12 of the solid formulation 10, 40, 60. At the beginning of the method, the portions 12 are secured to one another by the presence of the support tape 124.

As shown in FIG. 3, the portions 12 of the strip 120 are aligned along the longitudinal X and transverse Y directions. Along the transverse direction, the strip 120 for example includes between three and one hundred aligned portions 12.

The printing device 104 includes a first 130 and second 132 printing module, respectively intended to deposit the first 14 and second 16 compositions on the substrate 12.

Each printing module 130, 132 includes: a printhead 134; a reservoir 136; a pipe 138 connecting said head to said reservoir; and a pump 140 arranged on said pipe.

The reservoir 136 of each of the first 130 and second 132 printing modules respectively contains a first 144 and second 146 liquid formulation, respectively making it possible to obtain the first 14 and second 16 compositions. Preferably, the reservoir 136 is provided with a device for agitating the liquid formulation. Preferably, the manufacture of the liquid formulation 144, 146 is done directly in the reservoir 136, shortly before starting up the printing device 104.

According to an advantageous embodiment, the first liquid formulation includes a pH regulator to improve the preservation of the active ingredient. According to a possible embodiment (which can be described as static), the pH regulator is incorporated into the first liquid formulation, e.g. during its preparation, and at the latest when the reservoir is filled with the first liquid formulation. According to another embodiment (which can be described as dynamic), the pH regulator is added to the liquid formulation in the reservoir 136 after a pH measurement (e.g. using a pH probe in the reservoir). Depending on the result of the pH measurement, a pump (or other injection device) is activated to add an appropriate volume of acidic or basic solution to correct the pH. According to a possible embodiment, the method applies a pH adjustment technique similar to any one of the following for adjusting the spraying tuning (by using one or more pH thresholds, which may represent positive or negative deviations from the desired pH, each threshold being usable to trigger an adjustment action).

The printhead 134 of each of the first 130 and second 132 printing modules is able to spray microdroplets of liquid formulation 144, 146 on the film 18. The printhead 134 is preferably a printhead of the thermal or piezoelectric type, more preferably piezoelectric. Such printheads are in particular marketed by the companies Konica Minolta and Fujifilm Dimatix.

According to an alternative that is not shown, each printing module further includes a buffer tank, inserted between the pipe 138 and the printhead 134. Said printhead is directly connected to the buffer tank.

Aside from the previously cited components of the first 14 or second 16 composition, the liquid formulation 144, 146 comprises at least one solvent and optionally excipients.

The solid formulation 10, 40, 60 being intended to be adjusted, the at least one solvent of the liquid formulation 144, 146 preferably has little to no toxicity, as for example a class 3 ICH. Likewise, the at least one solvent of the liquid formulation 144, 146 preferably has a low boiling temperature, in order to evaporate easily after printing. The at least one solvent for example includes water and/or ethanol. Optionally, the at least one solvent further comprises dimethylsulfoxide (DMSO) for better solubilization of the compounds.

Furthermore, in order to optimize the precision of the printing, it is preferable to check the viscosity and/or the surface tension of the first 144, and optionally second 146, liquid formulations.

Preferably, the viscosity of the first 144 and/or the second 146 liquid formulation is between 2 mPa·s and 20 mPa·s, more preferably between 2 mPa·s and 10 mPa·s. Preferably, the surface tension of the first 144 and/or the second 146 liquid formulation is between 20 mN/m and 50 mN/m, and, according to a possible embodiment, between 25 mN/m and 50 mN/m.

A sufficient viscosity and surface tension allow the creation of stable microdroplets, i.e., that do not split during spraying by the printhead 134.

As a result, the first 144 and/or second 146 liquid formulation preferably comprises an additive to change its viscosity and/or its surface tension, such as propylene glycol.

Furthermore, the first 144 and/or second 146 liquid formulation preferably comprises a pH regulator additive, chosen based on the stability characteristics of the active ingredient.

This may be an acid additive such as acetic acid, or a basic additive.

At the output of the printing device 104, the apparatus 100 comprises a conveyor 150 able to receive the strip 120 of film; and optionally a rotary roller 152 making it possible to wind the support tape 124 to separate it from said strip 120. In an alternative embodiment where the strip 120 is not precut, the apparatus further comprises a device for cutting the strip between the drying device and the analysis device.

The conveyor 150 for example includes an endless strip 154 extending between two drive cylinders.

The drying device 106 is arranged on the path of the conveyor 150. Said drying device 106 is of the furnace type and comprises a heating element such as an electric resistance, or an infrared radiation element.

The analysis device 108 is also arranged on the path of the conveyor 150, downstream from the drying device 106 and above the endless strip 154. The analysis device 108 is capable of measuring a quantity of first active ingredient supported by the substrate 12 of a solid formulation 10, 40, 60, said measurement not destroying said formulation. The analysis device 108 preferably includes an electromagnetic spectroscopy device, of the near infrared spectroscopy (NIR spectroscopy) type, or Raman spectroscopy type, or a hyperspectral imaging device. All these devices perform, in a known manner, measurements that are non-destructive in that they only subject the active ingredient to spectroscopy (which in principle lasts no longer than one minute), which does not alter it. Other types of measurement are also possible, such as weighing by a precision balance and more generally technologies called PAT (“Process Analytical Technologies”).

These measurements, which determine a (precise) quantity of the active ingredient, allow comparing this quantity to a reference value and thus assessing the difference between the desired quantity (reference value) and the actual quantity. According to an advantageous embodiment, the measurement is carried out after the liquid spray formulation has dried substantially, so that the amount of solvent still present is small and distorts the measurement as little as possible. Even more advantageously, the measurement is carried out after a step of complete drying. Indeed, experiments have shown that the more advanced the drying process, the more accurate the measurement. Drying stabilizes the formulation and increases its concentration.

According to a possible embodiment, the method computes the deviation between the measured quantity and the reference value, in relative form Eᵣ (e.g. as a percentage). Alternatively, the method computes this deviation in absolute form Eₐ, e.g. in volume or mass, e.g. in microlitres (μL) or milligrams (mg), or in any other appropriate unit.

According to a possible embodiment, the method defines a threshold above which the quantity of active ingredient is considered inaccurate. This threshold can be expressed in relative Sᵣ form, for example by means of a percentage (such as: Sᵣ=1.5%). This threshold is alternatively expressed in absolute form Sₐ, for example in volume or mass (such as: Sₐ=3 μL, or Sₐ=4 mg).

According to a possible embodiment, when the method determines that the difference between the quantity of active ingredient measured in a given portion deviates from the reference value by more than the threshold (for example, more than the 1.5% difference Eᵣ, or more than the 3 μL difference Eₐ, or more than the 4 mg difference Eₐ), it triggers an action. For example, if the reference value is 400 mg, and the threshold is 1.5%, the method triggers an action as soon as the quantity exceeds 406 mg or falls below 394 mg (i.e., 1.5% above or below 400 mg). Similarly, for a threshold of 4 mg, the process triggers an action as soon as the quantity exceeds 404 mg or falls below 396 mg.

According to a possible embodiment, this action consists of triggering a spraying adjustment action. According to a possible embodiment, the adjustment consists in changing the resolution (expressed for example in DPI, i.e. dots per inch) of the spraying device (which may be close to inkjet printer technologies). Adjustment can be made more generally by modifying a spraying control. If the threshold has been exceeded upwards, spraying is reduced for subsequent stages of continuous manufacture (i.e. for subsequent portions—not for the portion that was the subject of the measurement that triggered the adjustment action).

Similarly, if the threshold has been exceeded downwards, the spraying is increased for subsequent stages of continuous manufacture.

According to another possible embodiment (for which the thresholds may be higher than the illustrative numerical values mentioned above, e.g. Sᵣ=10%) this action consists in sorting the portion that gave rise to the inaccurate measurement. This sorting consists, for example, in routing the portions with inaccurate measurements to a receptacle that can be disposed of when it is full (destruction). Alternatively, in the event that the portion contains an insufficient quantity of active ingredient, the method may, instead of discarding the portion, re-route it once again through a spraying step in order to supplement the quantity of active ingredient and bring it closer to the reference value.

According to another possible embodiment, this action consists in signaling the existence of a deviation via an electronic warning device (leading, for example, to the lighting of a red LED, the display of a message in the form of a text or pictogram, the sounding of an alarm, the sending of an automatic e-mail, etc.). According to a possible embodiment, the alarm is triggered continuously as long as the measurements do not return within the limit defined by the threshold. This alarm can be cumulated with the action of sorting (and if necessary renewed spraying) and can also be cumulated with the action of adjusting the spraying.

According to a possible embodiment, the method defines several thresholds, with different actions being triggered depending on the thresholds crossed. According to a possible embodiment, the process includes a low threshold that triggers a first action, and a high threshold that triggers a second action. These thresholds can be relative or absolute. For example, the relative low threshold is 1.5% and the relative high threshold is 10%, or the absolute low threshold is 4 mg and the absolute high threshold is 20 mg. The choice of threshold values depends on the active ingredient and may depend on personalized parameters (age, sex, height, weight, medical history, etc.). This choice can be made by a person qualified in pharmacy and thus lead to a definition of different thresholds for each of the many possible configurations. According to a possible embodiment, the method is then set to automatically select the relevant threshold(s) thus defined according to input data (active ingredient, age, sex, height, weight, medical history, etc.). According to a possible embodiment, the method includes means for manually entering the threshold(s), the latter replacing the automatic selection (if the latter is implemented), which is thus deactivated by the manual entry.

According to a possible embodiment, the first action is a signaling action (alarm) of the low threshold being exceeded. According to a possible embodiment, the first action is a spraying adjustment action (already described). According to a possible embodiment, exceeding the low limit triggers the two above-mentioned actions (reporting and spraying adjustment).

According to a possible embodiment, the second action is a warning (alarm) action signaling that the high threshold is exceeded, which may be different from the warning (alarm) action signaling that the low threshold is exceeded (to indicate the increased importance of the alarm). According to a possible embodiment, the second action is a sorting action and, optionally involves an additional spraying (already described). According to a possible embodiment, exceeding the high threshold triggers the two above mentioned actions (reporting and sorting, plus possibly additional spraying).

According to a variant, the method defines a different threshold upwards and downwards (indeed, overdosing and underdosing of a drug does not necessarily have the same consequences). Thus, the method defines, according to a possible embodiment, a low upper threshold and a high upper threshold, as well as a low lower threshold and a high lower threshold. Each of these thresholds may be relative or absolute.

According to a possible embodiment, the same action is triggered when the low lower threshold is exceeded and when the low upper threshold is exceeded. But according to a variant, they are different actions; for example, the alarm can be different so that an operator is informed of the direction in which the threshold is exceeded, or in yet another variant, the adjustment can be different (for example faster in case of overdosing than in case of underdosing, or vice versa).

According to a possible embodiment, the same action is triggered when the high lower threshold is exceeded and when the high upper threshold is exceeded. But according to a variant, these are different actions; for example, the alarm may be different so that an operator is informed of the direction in which the threshold is exceeded, or in yet another variant, as already mentioned, the sorting may be different (it may lead to discarding an overdosed portion, but trying to “fix” an underdosed portion rather than discarding it).

According to a possible embodiment, the method implements, in a continuous manner, an action of adjustment of the spraying tuning, without checking whether a threshold is exceeded. For example, the method implements a feedback loop, whereby a percentage of the difference between the measured quantity and the reference value is subtracted from the spraying tuning value to be submitted as an input parameter to the subsequent spraying command (and the resulting modified spraying tuning is submitted to the spraying command). This percentage can take various values. The higher the percentage, the faster the correction, but the greater the risk of overcorrecting (and thus generating a deviation in the other direction). The lower the percentage, the slower the adjustment, but the less likely it is to generate parasitic oscillations. For example, if the spraying tuning is (at least initially) equal to the reference value, if the percentage is set to 30%, if the reference value is set to 200 mg, and the measured amount is 210 mg, the deviation Eₐ is 10 mg. The method then sends the sprayer an instruction to spray 3 mg less (−30% of 10 mg). If in the next iteration the measured quantity (e.g. 207 mg) is still greater than the reference value (in the example given, Eₐ=7 mg), the method instructs the sprayer to spray 2.1 mg less (−30% of 7 mg). In this manner, the method updates, during iterations, the spraying tuning value, in order to bring the measured value (after spraying) closer to the reference value. If the spraying tuning value is not equal to the reference value (at the beginning of the process) but is only representative of it (e.g. if this tuning value is a DPI print resolution), the method adjusts (according to a possible implementation) the tuning value proportionally to the deviation found. For example, if the measured value is 10% below the reference value, and if the percentage applied for adjustment is (as above) 30%, the tuning value is increased by 30% of 11.11% i.e. by 3.33% (N.B. for a value that is 10% below normal to return to normal, it must be increased by 11.11%). For a tuning value that does not vary linearly compared to the measured quantity, an appropriate non-linear adjustment can be applied (i.e. different from the above-mentioned proportional adjustment).

According to another possible embodiment, an adjustment action of the spraying tuning is triggered only in response to a threshold being exceeded. In this case, the measured quantity must be sufficiently different from the predetermined value for an adjustment to be triggered. This adjustment can be a feedback loop as described in the previous paragraph. But it can also be another type of correction. For example, each time it is assessed that a threshold is exceeded, the method may adjust the spraying command by a constant value which is lower than or equal to the threshold (i.e. not by a percentage of the deviation). Indeed, it is certain that the deviation is greater than or equal to this constant value.

In a simplified implementation, there is no adjustment of the spraying command. The adjustment is carried out once and for all at the start of each production run, and the comparison of the measured quantity and the reference value is then not used to trigger an adjustment (but to trigger other actions, e.g. sorting of portions).

The sorting device 110 is also arranged on the path of the conveyor 150, downstream from the analysis device 108. The sorting device 110 is capable of removing the solid formulations 10, 40, 60 from the conveyor that do not have satisfactory analysis results. The sorting device 110 for example includes a nozzle 156, which blows compressed air or suctions the defective solid formulations 10, 40, 60 to remove them from the conveyor 150. Such a sorting is advantageous, as it allows an exhaustive control of the manufactured products. Any product resulting from the method is then of adequate quality (since it has been checked). This makes it possible to release the final product directly at the end of the manufacturing process and dispense it, without delay (i.e. without having to implement an additional quality procedure), to patients.

The packaging device 111 is arranged at the output of the conveyor 150. Upstream from the packaging device, two rollers 160 are arranged, each roller being able to empty a sheet of packaging material 162 of the aluminum sheet type covered with a plastic film. The packaging device 111 includes driving means able to arrange a sheet of packaging material 162 in contact with each of the faces of the solid formulations 10, 40, 60.

Preferably, the packaging device 111 comprises an inscription device 164, of the laser etching or printer type, capable of inscribing information on the packaging material 162 as it scrolls.

Downstream from the rollers 160, the packaging device 111 comprises a sealing device 166, capable of fastening the two sheets of packaging material 162 to one another, so as to form a closed compartment 170 around each of the solid formulations 10, 40, 60. The sealing device 166 for example includes a heating element. Preferably, the sealing device 166 also includes means for implementing the sealing under a controlled atmosphere, of the nitrogen type.

Downstream from the sealing device 166, the packaging device 111 includes a cutting device 168 capable of separating the closed compartments 170 from one another that contain the solid formulations 10, 40, 60.

A method 200 for manufacturing a solid formulation 10, 40, 60 will now be described. This method, diagrammed by the logic chart of FIG. 4, is implemented by the program 114 stored in the electronic control unit 112.

First (step 202), the precut strip 120 of film 18, arranged on the support tape 124, is presented at the inlet of the printing device 104 and moves along the longitudinal direction X. The printhead 134 of the first printing module 130 deposits, on the first face 20 of each portion 12 of the strip 120, microdroplets of first liquid formulation 144 (step 204). The deposition is done on a first zone 26, 46, 66 of each portion 12. The quantity of first liquid formulation 144 per portion 12 is managed by the program 114, as a function of the quantity of first active ingredient desired (reference quantity) for the solid formulation 10, 40, 60 manufactured. The apparatus 100 is, for example, calibrated before implementing the manufacturing method, by assaying the first liquid formulation 144 in the first active ingredient.

In the case of the second and third embodiments, the printhead 134 of the second printing module 132 next deposits, on the second zone 48, 68 of each portion 12 of the strip 120, microdroplets of second liquid formulation 146 (step 206). The quantity of second liquid formulation 146 per portion 12 is managed by the program 114.

At the outlet of the printing device 104, the support tape 124 is wound on the rotary roller 152 and separated from the strip 120 of film 18. The portions 12 of said strip 120 are received on the conveyor 150 and driven to the drying device 106. The solvents of the first 144 and second 146 liquid formulations finish evaporating (step 208), leading to the first 14 and second 16 compositions being deposited on the substrate 12. The solid formulation 10, 40, 60 previously described is thus obtained.

The solid formulations 10, 40, 60 are next driven by the conveyor to the analysis device 108. For each solid formulation 10, 40, 60, said analysis device measures the quantity Q of first active ingredient deposited on the substrate 12 (step 210). Said measurement is, for example, done by near infrared spectroscopy or Raman spectroscopy.

The program 114 compares each measured value Q with a reference value Q_{ref}, stored in said program (step 212). For example, the program 114 checks whether the value Q is within a range [Q_{ref}±Δ_{Q}], Δ_{Q} being a value also stored in said program (corresponding to a single threshold). According to a possible embodiment, the program 114 adjusts the spraying tuning to the Q value (this is symbolized in FIG. 4 by the inclined arrow pointing from step 212 to spraying step 204). According to a possible embodiment, the program adjusts the spraying tuning only if Q deviates from Q_{ref} by more than Δ_{Q} (whether downwards or upwards). According to a variant, one (or more) specific threshold(s) is (are) used. According to another variant, the adjustment is systematic (regardless of the value of Q, and thus independent of any threshold).

The program 114 identifies each solid formulation 10, 40, 60 as belonging to a first or a second group, the value Q of which respectively is within or is not within the targeted range. The sorting device 110 removes, from the conveyor 150 (step 214), the formulations belonging to said second group.

Downstream from the sorting device 110, the solid formulations 10, 40, 60 remaining on the conveyor 150 therefore all comprise a quantity Q of first active ingredient within the range [Q_{ref}±Δ_{Q}].

In the case where the second composition 16 comprises a second active ingredient, the analysis and sorting steps are also carried out on said second active ingredient (step 216).

Each solid formulation 10, 40, 60 is next packaged individually by the packaging device 111 (step 218). On each package, the inscription device 164 inscribes information such as a lot number, an identifier of the first and/or second active ingredient, or a manufacturing or expiration date.

Solid formulations 10, 40, 60, packaged individually, are thus recovered at the output of the apparatus 100.

Optionally, the method further comprises a secondary packaging step, in which solid formulations 10, 40, 60 packaged individually are grouped together in a container of the cardboard box type.

The apparatus 100 and the method 200 previously described allow significant flexibility in the production of solid formulations 10, 40, 60. The program 114 makes it possible to adjust, vary and monitor, in real time, the quantity of first 144 and/or second 146 liquid formulation sprayed on each portion 12 of the strip 120. It is thus easy to successively produce solid formulations 10, 40, 60 corresponding to different dosages, without interrupting the production.

Likewise, it is possible to modify the liquid formulation 144, 146 of the printing device 104 quickly, so as to produce continuously, from a same substrate, solid formulations comprising different active ingredients.

The apparatus 100 and the method 200 thus make it possible to produce medicinal drugs easily and at a low cost, by adapting the dose to each patient.

In the embodiment described above, the provided strip 120 is precut before the printing step 204. In an alternative that is not shown, the provided strip 120 is continuous and the method comprises, after the printing step, a step for cutting said strip into a plurality of portions 12. Preferably, said cutting step is carried out before the analysis 210, 212 and sorting 214 step.

According to an alternative embodiment, the solid formulations 10, 40, 60 are not orodispersible formulations, but are intended to be used in the form of buccal patches, which may or may not be mucoadhesive. Only the nature of the substrate 12 must be modified to produce this alternative.

The embodiments described for the implementation of the method may be transposed to the embodiments relating to the corresponding manufacturing apparatus, and vice versa.

### EXAMPLE EMBODIMENTS

1.—Production of the Film 18 of the Substrate 12

The aim is to obtain a flexible and non-brittle substrate 12 with a slightly acidic pH (4.7-5.0) with a low humidity level (in particular below 10%), and that dissolves in less than 10 s. The four formulations below have been developed:

→Formulation 1:
Hypromellose
Plant glycerin
Purified water
Diluted phosphoric acid R

→Formulation 2:
Pullalane
Microcrystalline cellulose
Cornstarch
Polysorbate 80
Plant glycerin
Purified water
Diluted phosphoric acid r
Titanium (IV) oxide

→Formulation 3:
HPMC (Hydroxypropyl Methylcellulose)
Citric Acid
Kollicoat® (Sigma-Aldrich)

→Formulation 4:
HPMC
Citric acid
Kollicoat®
Moisture protection agent such as EPO (Poly(butyl methacrylate-co-(2-demethylaminoethyl) methacrylate-co-methyl methacrylate) 1:2:1)

Preferably, the cellulose derivatives represent at least 40 wt % of the dry composition; the acid is preferably used in a proportion of less than 1%.

The formulation is spread over a flat surface and dried. The film 18 is obtained with a thickness of about 250 μm. Alternatively, it is obtained with a smaller thickness, e.g. 100 μm or 120 μm.

2.—Manufacturing of the First Liquid Formulation 144
Active ingredient (Desmopressin)—40 mg/ml
DMSO: Ethanol (50:50)
Propylene Glycol 30%
Acetic acid 1%
Dye (for example Sunset Yellow)—5 mg/ml

1.-15. (canceled)

16. A method for manufacturing a solid formulation for oral administration, said solid formulation comprising an edible substrate portion on which a first composition comprising a first pharmaceutical active ingredient is deposited, said method comprising:
providing an edible substrate in film form, wherein the film comprises a plurality of portions, each designed to support a quantity Q of the first pharmaceutical active ingredient corresponding to predetermined value Q_{ref};
spraying onto a portion of the edible substrate a first liquid formulation comprising a first pharmaceutical active ingredient and a first solvent;
evaporating the first solvent so as to form a first solid composition deposited on the portion of the edible substrate;
measuring a quantity Q of the first pharmaceutical active ingredient supported by the portion of the edible substrate without destroying the solid composition, and comparing the measured quantity Q to a predetermined value Q_{ref}, and
dividing the edible substrate comprising deposited solid composition into the plurality of portions, thereby obtaining a plurality of said solid formulations for oral administration.

17. The method according to claim 16, further comprising:
upon identifying an abnormal deviation between a measured quantity Q and the predetermined value Q_{ref}, reporting a problem with tuning of the spraying.

18. The method according to claim 16, further comprising:
adjusting tuning of the spraying according to the comparison between a measured quantity Q and the predetermined value Q_{ref}.

19. The method according to claim 16, further comprising:
classifying the portions of the plurality of portions into two groups based on the comparison between the measured quantity Q and the predetermined value Q_{ref}, and separating the two groups from one another.

20. The method according to claim 16, wherein the first liquid formulation has a viscosity between 2 mPa·s and 20 mPa·s.

21. The method according to claim 16, wherein the first liquid formulation has a surface tension between 25 mN/m and 50 mN/m.

22. The method according to claim 16, wherein the first pharmaceutical active ingredient is selected from an analgesic, an antihistamine, an anti-inflammatory, an antiepileptic, and a natural, synthetic or biotechnologically-produced hormone.

23. The method according to claim 16, wherein the first liquid formulation further comprises a dye.

24. The method according to claim 16, wherein the first liquid formulation further comprises a pH regulator.

25. The method according to claim 16, wherein the edible substrate comprises one or more hydrolysable polymers.

26. The method according to claim 16, wherein the edible substrate comprises one or more cellulose derivatives selected from hydroxypropyl cellulose, hydroxypropyl methylcellulose, carboxymethylcellulose sodium, and hydroxyethyl cellulose.

27. The method according to claim 16, wherein the edible substrate comprises a moisture protection agent.

28. The method according to claim 16, wherein the edible substrate has a pH adapted to the active ingredient.

29. The method according to claim 16, wherein, in the step of providing the edible substrate in film form, the film is a precut film comprising the plurality of portions.

30. The method according to claim 16, wherein:
in the step of providing edible substrate in film form, the film is a continuous film; and
the dividing step comprises cutting the film into the plurality of portions and separating the portions from each other.

31. The method according to claim 16, wherein:
the step of spraying the first liquid formulation onto a portion of the edible substrate comprises spraying the first liquid formulation onto a first zone of a portion of the edible substrate; and the method further comprises:
spraying a second liquid formulation onto a second zone of the portion of the edible substrate separate from the first zone, the second liquid formulation comprising a second solvent and one or more of (i) a second pharmaceutical active ingredient and (ii) a flavoring, and
evaporating the second solvent,
such that each portion of the edible substrate comprises a first solid composition on a first zone and a second solid composition on a second zone separate from the first zone.

32. A solid formulation for oral administration made by the method according to claim 16.

33. A solid formulation for oral administration made by the method according to claim 31.

34. An apparatus for manufacturing a solid formulation for oral administration, said solid formulation comprising an edible substrate portion on which a first composition comprising a first pharmaceutical active ingredient is deposited, said apparatus comprising:
a roller configured to provide a film comprising a plurality of edible substrate portions;
a printer configured to spray, onto an edible substrate portion, a first liquid formulation comprising a first pharmaceutical active ingredient and a first solvent;
a dryer configured to evaporate the first solvent so as to form a first solid composition deposited on the edible substrate portion;
a nondestructive measuring device configured to measure a quantity Q of the first pharmaceutical active ingredient supported by an edible substrate portion without destroying the solid composition; and
an electronic controller programmed to compare a measured quantity Q with a predetermined value Q_{ref} and control the printer to spray the first liquid formulation onto an edible substrate portion.

35. The manufacturing apparatus according to claim 34, further comprising a packaging device configured to individually package an edible substrate portion on which a solid composition is deposited, thereby obtaining a packaged solid formulation for oral administration.
